(19) United States
(12) Patent Application Publication
(10) Pub. No.: US 2013/0090377 A1
(43) Pub. Date: Apr. 11, 2013

(54) COMPOSITION COMPRISING COUMESTROL OR A BEAN EXTRACT CONTAINING COUMESTROL

(71) Applicants: Hee Young Jeon, Gyeonggi-do (KR); Si Young Cho, Seoul (KR); Hyun Woo Jeong, Seoul (KR); Dae Bang Seo, Gyeonggi-do (KR); Sang Jun Lee, Gyeonggi-do (KR)

(72) Inventors: Hee Young Jeon, Gyeonggi-do (KR); Si Young Cho, Seoul (KR); Hyun Woo Jeong, Seoul (KR); Dae Bang Seo, Gyeonggi-do (KR); Sang Jun Lee, Gyeonggi-do (KR)

(73) Assignee: AMOREPACIFIC CORPORATION, Seoul (KR)

(21) Appl. No.: 13/637,962

(22) PCT Filed: Mar. 31, 2011
(86) PCT No.: PCT/KR2011/002222

§ 371 Date: Dec. 7, 2012

(30) Foreign Application Priority Data
Mar. 31, 2010 (KR) 10-2010-0029170

(51) Int. Cl. A61K 31/35

(52) U.S. Cl. CPC A61K 31/35
USPC 514/453

(57) ABSTRACT

The present invention relates to a composition for activating mitochondria comprising coumestrol or a bean extract containing coumestrol.

### TECHNICAL FIELD

The present disclosure relates to a composition for activating mitochondria.

### BACKGROUND ART

Mitochondria are organelles found in most eukaryotic cells and have their own mitochondrial DNA (mtDNA) which is distinct from the nuclear DNA. The main function of mitochondria is the production of ATP used as an intracellular source of energy. ATP is generated by the electron transport chain using NADH and FADH₂ generated by the TCA cycle occurring in the mitochondrial matrix. Thus produced ATP is used in various biosynthetic and metabolic processes requiring energy.

Also, mitochondria store calcium ions which play an important role in intracellular signaling in the matrix and supply them to the cytoplasm. Further, they are known to play a central role in apoptosis and regulation of cellular proliferation and metabolism.

The mitochondrial DNA (mtDNA) is relatively vulnerable to damage since it lacks repair mechanism and the protein histone that protects DNA, unlike the nuclear DNA. Damage to the mitochondrial DNA leads to decreased functions of mitochondria and causes decreased synthesis of ATP, which is the source of energy required for cellular activities, and various mitochondrial diseases.

### DISCLOSURE

### Technical Problem

The present disclosure is directed to providing a composition for activating mitochondria.

### Technical Solution

In an aspect, the present disclosure provides a composition for activating mitochondria comprising coumestrol or a bean extract comprising coumestrol as an active ingredient.

### Advantageous Effects

The composition according to an embodiment of the present disclosure, which comprises coumestrol or a bean extract comprising coumestrol as an active ingredient, may activate SIRT1 and increase the amount of intracellular mitochondria. Accordingly, the composition may provide an effect of relieving fatigue, improving endurance, improving muscular strength or improving motor function by promoting energy metabolism, and may prevent or improve obesity, hyperlipidemia or hypertension by reducing body fat. Also, the composition may prevent or improve mitochondrial diseases.

### DESCRIPTION OF DRAWINGS

FIG. 1 shows increase of SIRT1 activity by coumestrol.

FIG. 2 shows mitochondria-associated genes whose expression is increased by coumestrol.

FIG. 3 shows increase of mitochondria in muscle cells by coumestrol.

### MODE FOR INVENTION

As used herein, “extract” means a substance extracted from a natural substance, regardless of extraction method or ingredients. The term is used in a broad sense including, for example, ingredients soluble in water or an organic solvent extracted from a natural substance using the solvent, or specific ingredients of a natural substance such as oil extracted therefrom.

Hereinafter, the present disclosure is described in further detail.

In an aspect, the present disclosure provides a composition for activating mitochondria comprising coumestrol, a natural substance comprising coumestrol or an extract thereof as an active ingredient.

Coumestrol (CMS; 3,9-dihydroxy-6H-benzofuro(3,2-c)(1)benzopyran-6-one) has a structure of Chemical Formula 1:

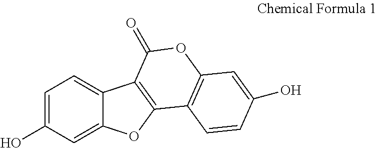 [SMILES: O=c1oc2cc(O)ccc2c2oc3cc(O)ccc3c12]

Coumestrol is one of phytoestrogens and is usually found in the seed, root or leaf of plants in the family Leguminosae or Compositae. It can be classified as a coumestan-like isoflavonoid and is known to have an estrogenic effect.

In an exemplary embodiment of the present disclosure, the composition comprises coumestrol, a natural substance comprising coumestrol or an extract thereof. In another exemplary embodiment of the present disclosure, the natural substance comprising coumestrol may be one or more bean selected from soybean, pea, mung bean and sprouted beans sprouted therefrom, red clover, alfalfa, Brussels sprout, or the like. In another exemplary embodiment of the present disclosure, the natural substance comprising coumestrol may be bean.

In an exemplary embodiment of the present disclosure, the bean may be any plant comprising coumestrol in the family Leguminosae, without particular limitation. For example, the ban that can be used in the present disclosure may be one for bean paste, bean curd, bean sprout, rice cooking or green bean. The bean varieties for bean paste or bean curd include daepung, hojang, jangwon, daehwang, sodam, songhak, daewon, jinpum, danbaek, duyu, shinpaldal, taegwang, manli, jangsu, muhan, baegun, saeal, hwangkeum and jangyeop. The bean varieties for bean sprout include shinhwa, sowon, anpyeong, seonam, dachae, sorok, soho, somyeong, dawon, pungsan-namul, iksan-namul, sobaek-namul, gwangan, danyeop and eunha. The bean varieties for rice cooking include cheongja, heukcheong, galmi, seonheuk, geomjeong-kong and ilpumgeomjeong-kong. And, the bean varieties for green bean include daol, shinrok, saeul, geomjeongeul, seokryang-putkong, hwaeom-putkong and keuneul. In another exemplary embodiment of the present disclosure, the bean may be one that can be sprouted and is resistant to damage from disease and harmful insects. Such bean varieties include, for example, shinhwa, sowon, anpyeong, seonam, dachae, sorok, soho, somyeong, dawon, pungsan-namul, iksan-namul, sobaek-namul, gwangan, danyeop and eunha.

In an exemplary embodiment of the present disclosure, the natural substance or the extract thereof may comprise a large quantity of coumestrol. Specifically, it may comprise 0.01-50 wt %, more specifically 0.1-30 wt %, of coumestrol based on the total weight of the natural substance or the extract thereof.

In an exemplary embodiment of the present disclosure, the extract of the natural substance comprising coumestrol may be obtained by extracting the natural substance comprising coumestrol with water or ethanol at normal or elevated temperature, completely concentrating the resulting extract and dispersing again in water, and fractionating with one or more solvent of equal volume selected from hexane, dichloromethane, chloroform, ethyl acetate, butanol, ethanol, methanol and water. However, the extraction method is not limited thereto and any extraction method may be employed.

As described above, mitochondria synthesize ATP which is used as the basic source of energy required for life activities by breaking down glucose through cellular respiration. Accordingly, abnormal functioning of mitochondria leads to cell death and negative effects on tissues and organs. The abnormal functioning of mitochondria also causes disorders of nerve and muscle tissues and metabolic diseases. It is also a cause of aging.

The coumestrol included in composition according to an embodiment of the present disclosure as an active ingredient may promote the mitochondrial activity by increasing the activity of sirtuin 1 (SIRT1). SIRT1 is an NAD-dependent deacetylase and is involved in the production of mitochondria.

The composition according to an embodiment of the present disclosure, which comprises coumestrol as an active ingredient, may promote energy metabolism by activating SIRT1 and mitochondria. Through this, it may provide an effect of relieving fatigue, improving endurance, improving muscular strength or improving motor function. Further, the composition may prevent or improve obesity, hyperlipidemia or hypertension since it reduces body fat by increasing energy consumption in the body.

The composition according to an embodiment of the present disclosure, which comprises coumestrol as an active ingredient, may prevent or improve mitochondrial diseases.

In an exemplary embodiment of the present disclosure, the mitochondrial disease comprises one or more of a neuropsychiatric disease, a cardiovascular disease, a visceral disease, an endocrine disease, a sensory system disease, a joint disease and a muscular disease caused by decreased mitochondrial activity.

In an exemplary embodiment of the present disclosure, the neuropsychiatric disease comprises one or more of dementia, Parkinson's disease, stroke, neuropsychiatric disorder, autism, mental retardation, seizure, stroke-like seizure, migraine and nerve pain, but is not limited thereto. These diseases may occur owing to accumulation of reactive oxygen species or disorder of nerve tissues caused by decreased mitochondrial activity.

In an exemplary embodiment of the present disclosure, the cardiovascular disease comprises one or more of cardiac dysfunction, heart failure, cardiomyopathy, tachycardia, sideroblastic anemia, Pearson syndrome, refractory anemia and hypotension, but is not limited thereto. These diseases may occur owing to calcium overload that may cause cardiac dysfunction or excessive oxidative stress as a result of decreased mitochondrial activity.

In an exemplary embodiment of the present disclosure, the visceral disease comprises one or more of liver failure, intestinal pseudoobstruction, irritable bowel syndrome, gastroesophageal reflux disease, constipation, diarrhea, renal tubular acidosis and Fanconi syndrome, but is not limited thereto. These diseases may occur owing to increased oxidative stress accompanying damage to the mitochondrial respiratory system caused by decreased mitochondrial activity.

In an exemplary embodiment of the present disclosure, the endocrine disease comprises one or more of exocrine pancreatic insufficiency, hypothyroidism, hypoparathyroidism, hypogonadism, developmental delay, type 2 diabetes and hypoglycemia, but is not limited thereto. These diseases may occur owing to endocrine disorder caused by mitochondrial dysfunction.

In an exemplary embodiment of the present disclosure, the sensory system disease comprises one or more of optic atrophy, strabismus, retinitis pigmentosa, blindness and hearing loss, but is not limited thereto. These diseases may occur owing to the abnormality in the sensory system caused by mitochondrial dysfunction.

In an exemplary embodiment of the present disclosure, the joint disease comprises one or more of degenerative arthritis, rheumatoid arthritis and osteoarthritis, but is not limited thereto. These diseases may occur owing to increased expression of inflammatory factors such as cyclooxygenase 2 (COX-2) in cartilage cells caused by decreased mitochondrial activity.

In an exemplary embodiment of the present disclosure, the muscular disease comprises one or more of ataxia, dysarthria, dysphagia, spasticity, encephalomyopathy, muscular pain, muscular dystrophy, muscle spasm, ptosis, ophthalmoplegia, progressive external ophthalmoplegia, Leber's hereditary optic neuropathy, breathing disorder, declined reflex and autonomic imbalance, but is not limited thereto. These diseases mainly occur owing to muscle weakness, exercise intolerance due to muscle wasting, or fatigue caused thereby as a result of defect in muscular metabolism caused by decreased mitochondrial activity and suppressed ATP production.

The composition according to an embodiment of the present disclosure, which comprises coumestrol or a bean extract comprising coumestrol, may prevent or improve the above-described diseases caused by decreased mitochondrial activity by activating mitochondria and promoting production thereof.

The composition according to an embodiment of the present disclosure may comprise 0.001-30 wt %, specifically 0.01-10 wt %, more specifically 0.1-5 wt %, of coumestrol or a bean extract comprising coumestrol based on the total weight of the composition. When the coumestrol or the bean extract comprising coumestrol is included in the above-described range, the intended effect of the present disclosure can be adequately achieved while both stability and safety are satisfied and favorable cost-effectiveness may be achieved.

In an exemplary embodiment of the present disclosure, the composition comprising coumestrol or a bean extract comprising coumestrol may be a food composition. The food composition may be a health food composition.

In an exemplary embodiment of the present disclosure, the food composition may be formulated into various forms including, for example, powder, granule, tablet, capsule, drink, etc., but is not limited thereto.

Further, the food composition may comprise one or more additive as desired. Examples of the additive may include: concentrated or powdered juice of grape, apple, orange, lemon, pineapple, banana, pear, etc.; water-soluble or oil-soluble vitamin such as retinyl palmitate, riboflavin, pyridoxine, cyanocobalamin, ascorbic acid, nicotinamide, calcium pantothenate, folic acid, biotin, cholecalciferol, choline bitartrate, tocopherol, β-carotene, etc.; flavor such as lemon flavor, orange flavor, strawberry flavor, grape flavor, vanilla essence, etc.; amino acid, nucleic acid or a salt thereof such as glutamic acid, sodium glutamate, glycine, alanine, aspartic acid, sodium aspartate, inosinic acid, etc.; plant fiber such as polydextrose, pectin, xanthan gum, glucomannan, alginic acid, etc.; or mineral such as sodium chloride, magnesium sulfate, potassium chloride, magnesium chloride, magnesium carbonate, calcium chloride, dipotassium phosphate, monosodium phosphate, calcium glycerophosphate, sodium ferrous citrate, ammonium ferric citrate, ferric citrate, manganese sulfate, copper sulfate, sodium iodide, potassium sorbate, zinc, manganese, copper, iodine, cobalt, etc.

The composition according to an embodiment of the present disclosure comprising coumestrol or a bean extract comprising coumestrol may be a pharmaceutical composition.

The pharmaceutical composition may further comprise a pharmaceutical adjuvant such as antiseptic, stabilizer, hydrating agent, emulsifying accelerator, salt and/or buffer for controlling osmotic pressure, etc. or other therapeutically useful substance, and may be prepared into various formulations for oral or parenteral administration.

The formulation for oral administration may include, for example, tablet, pill, hard or soft capsule, liquid, suspension, emulsion, syrup, granule, or the like. These formulations may comprise, in addition to the active ingredient, a diluent (e.g., lactose, dextrose, sucrose, mannitol, sorbitol, cellulose or glycine) or a lubricant (e.g., silica, talc, stearic acid and magnesium or calcium salt thereof or polyethylene glycol).

Typical formulations for parenteral administration include isotonic aqueous solution or suspension for injection.

Determination of the dose of the active ingredient is within the level of those skilled in the art. A daily dose of the active ingredient may vary depending on various factors, including progress and stage of the disease to be treated, age and physical condition of the subject, presence or absence of complications, and so forth. In general, the composition according to the present disclosure may be administered once or twice a day, in an amount of 1-500 mg/kg, specifically 30-200 mg/kg. However, the described administration dose does not limit the scope of the present disclosure by any means.

The features and effects of the present disclosure will be described in detail through test examples. However, the following test examples are provided for illustrative purposes only and are not intended to limit the scope of the present disclosure.

### Test Example 1

### Evaluation of SIRT1 Activating Effect

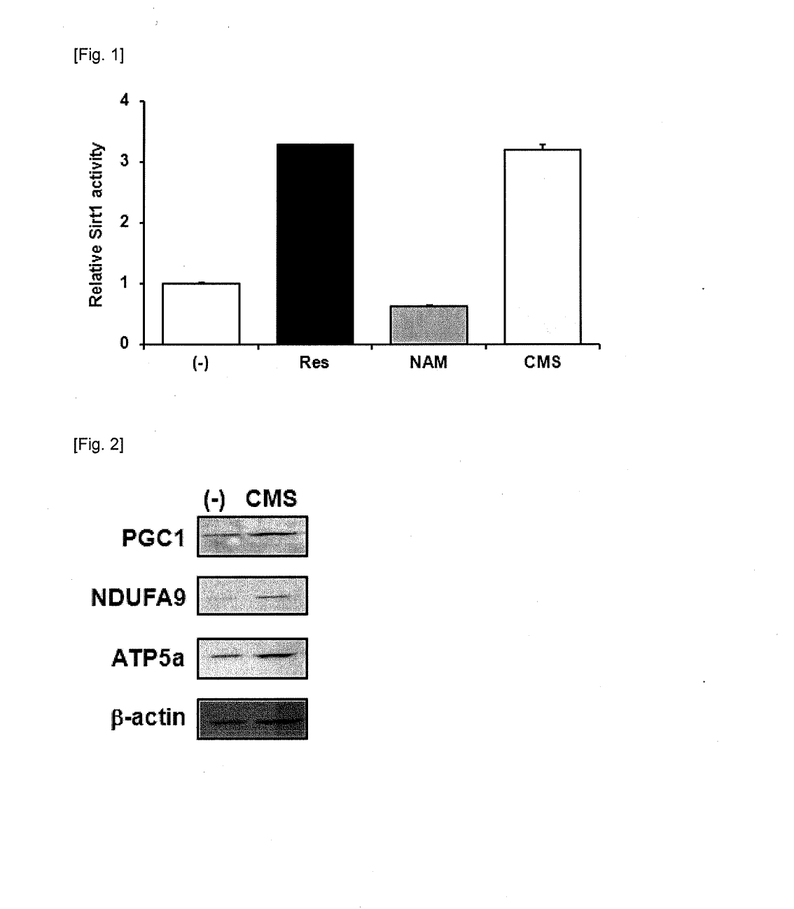

It was tested whether coumestrol can activate SIRT1 using the SIRT1 activity assay kit (Biomol). Cells were treated with 10 μM coumestrol. 50 M resveratrol (Res), which is known to activate SIRT1, was used as positive control and 1 mM nicotinamide (NAM), which is an inhibitor of SIRT1, was used as negative control. SIRT1 activity was calculated relative to the untreated group and is shown in FIG. 1.

As seen from FIG. 1, coumestrol (CMS) increased the activity of SIRT1, and the effect was comparable to that of resveratrol whose concentration was much higher than that of coumestrol. Thus, it seems that coumestrol has a much higher SIRT1 activating effect than resveratrol. Accordingly, a composition comprising coumestrol may inhibit aging and promote energy metabolism by activating SIRT1. Further it may relieve fatigue and improve endurance, muscular strength and motor function.

### Test Example 2

### Evaluation of Effect of Increasing Mitochondria in Muscle

C2C12 muscle cells derived from mouse (mouse myoblasts, ATCC) were differentiated for a week and then treated with 10 M coumestrol for 24 hours. Then, the expression of mitochondria-associated genes was confirmed at the protein level by western blotting. The result is shown in FIG. 2. The tested genes were PGC1 which induces mitochondria biosynthesis and NDUFA9 and ATP5a which are proteins that constitute the mitochondrial electron transport chain. As seen from FIG. 2, treatment with coumestrol resulted in remarkable increase of the expression of the genes as compared to the untreated group.

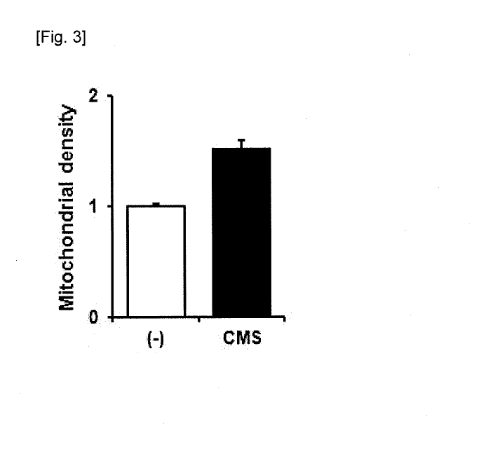

Also, the quantity of mitochondria in cells was measured by fluorescence after staining with MitoTracker Green (Invitrogen). The group treated with coumestrol showed increased mitochondria as compared to the untreated group (FIG. 3). As seen from FIG. 3, treatment with coumestrol resulted in remarkable increase of the quantity of mitochondria in cells.

Thus, it was confirmed that coumestrol can increase biosynthesis of mitochondria in muscle cells. Accordingly, a composition comprising coumestrol may improve motor function by increasing the amount of mitochondria in muscles and may prevent or improve mitochondrial myopathy. Further, it may reduce body fat by increasing energy metabolism in muscles and thus increasing energy consumption.

Formulation examples of the composition according to the present disclosure are described hereinafter. However, the scope of the present disclosure is not limited to the following examples.

### Formulation Example 1

### Soft Capsule

Coumestrol (80 mg), vitamin E (9 mg), vitamin C (9 mg), palm oil (2 mg), hydrogenated vegetable oil (8 mg), yellow beeswax (4 mg) and lecithin (9 mg) are mixed and prepared into a soft capsule filling solution according to a commonly employed method. 400 mg of the solution is filled per capsule. Separately from this, a soft capsule sheet is prepared using gelatin 66 (wt %), glycerin (24 wt %) and sorbitol (10 wt %), which is filled with the filling solution to prepare a soft capsule containing 400 mg of the composition according to the present disclosure.

### Formulation Example 2

### Tablet

Coumestrol (80 mg), vitamin E (9 mg), vitamin C (9 mg), galactooligosaccharide (200 mg), lactose (60 mg) and maltose (140 mg) are mixed and granulated using a fluidized bed dryer. After adding sugar ester (6 mg), the resulting composition (500 mg) is prepared into a tablet according to a commonly employed method.

### Formulation Example 3

### Drink

Coumestrol (80 mg), vitamin E (9 mg), vitamin C (9 mg), glucose (10 g), citric acid (0.6 g) and oligosaccharide syrup (25 g) are mixed. After adding purified water (300 mL), 200 mL of the resulting mixture is filled in a bottle. Then, a drink is prepared by sterilizing at 130° C. for 4-5 seconds.

### Formulation Example 4

### Granule

Coumestrol (80 mg), vitamin E (9 mg), vitamin C (9 mg), anhydrous crystalline glucose (250 mg) and starch (550 mg) are mixed, granulated using a fluidized bed granulator, and then filled in a pouch.

### Formulation Example 5

### Injection

| | |
|---|---|
| Coumestrol | 20 mg |
| Sterilized distilled water for injection | adequate |
| pH adjuster | adequate |

An injection is prepared using the above ingredients per ampule (2 mL) according to a commonly employed method.

Those skilled in the art will appreciate that the present disclosure may be changed and modified variously within the scope of the present disclosure.

1. A method for activating mitochondria comprising
administering coumestrol or a bean extract comprising coumestrol to a subject in such need in an amount effective to activate mitochondria.

2. The method according to claim 1, wherein the coumestrol or bean extract comprising coumestrol promotes energy metabolism.

3. The method according to claim 2, wherein the coumestrol or bean extract comprising coumestrol relieves fatigue, improves endurance, improves muscular strength or improves motor function.

4. The method according to claim 2, wherein the coumestrol or bean extract comprising coumestrol prevents or improves obesity, hyperlipidemia or hypertension by reducing body fat.

5. The method according to claim 1, wherein the coumestrol or bean extract comprising coumestrol prevents or improves mitochondrial disease.

6. The method according to claim 5, wherein the mitochondrial disease comprises one or more of a neuropsychiatric disease, a cardiovascular disease, a visceral disease, an endocrine disease, a sensory system disease, a joint disease and a muscular disease caused by decreased mitochondrial activity.

7. The method according to claim 6, wherein the neuropsychiatric disease comprises one or more of dementia, Parkinson's disease, stroke, neuropsychiatric disorder, autism, mental retardation, seizure, stroke-like seizure, migraine and nerve pain.

8. The method according to claim 6, wherein the cardiovascular disease comprises one or more of cardiac dysfunction, heart failure, cardiomyopathy, tachycardia, sideroblastic anemia, Pearson syndrome, refractory anemia and hypotension.

9. The method according to claim 6, wherein the visceral disease comprises one or more of liver failure, intestinal pseudoobstruction, irritable bowel syndrome, gastroesophageal reflux disease, constipation, diarrhea, renal tubular acidosis and Fanconi syndrome.

10. The method according to claim 6, wherein the endocrine disease comprises one or more of exocrine pancreatic insufficiency, hypothyroidism, hypoparathyroidism, hypogonadism, developmental delay, type 2 diabetes and hypoglycemia.

11. The method according to claim 6, wherein the sensory system disease comprises one or more of optic atrophy, strabismus, retinitis pigmentosa, blindness and hearing loss.

12. The method according to claim 6, wherein the joint disease comprises one or more of degenerative arthritis, rheumatoid arthritis and osteoarthritis.

13. The method according to claim 6, wherein the muscular disease comprises one or more of ataxia, dysarthria, dysphagia, spasticity, encephalomyopathy, muscular pain, muscular dystrophy, muscle spasm, ptosis, ophthalmoplegia, progressive external ophthalmoplegia, Leber's hereditary optic neuropathy, breathing disorder, declined reflex and autonomic imbalance.

14-15. (canceled)
